(19) United States
(12) Patent Application Publication
(10) Pub. No.: US 2026/0215777 A1
(43) Pub. Date: Jul. 30, 2026

(54) SURGICAL TISSUE FASTENER AND A DEVICE FOR APPLYING THE SAME

(71) Applicant: Gosai Gaurangkumar Ashwingiri, Gandhinagar (IN)

(72) Inventor: Gosai Gaurangkumar Ashwingiri, Gandhinagar (IN)

(21) Appl. No.: 19/130,851

(22) PCT Filed: Nov. 14, 2023
(86) PCT No.: PCT/IN2023/051055

§ 371 Date: May 16, 2025

(30) Foreign Application Priority Data
Nov. 16, 2022 (IN) 202221065571

(51) Int. Cl. A61B 17/064; A61B 17/00; A61B 17/068

(52) U.S. Cl. CPC A61B 17/0644; A61B 17/068; A61B 2017/00004; A61B 2017/0641

(57) ABSTRACT

The present disclosure proposes a surgical tissue fastener and a device for applying such fasteners to easily and effectively close wounds. The fastener comprises two tissue-capturing zones opposite each other substantially in a single plane. The surgical device comprises a tissue fastening assembly, a pair of tissue-approximating arms, a plurality of fasteners, a fastener deforming mechanism, at least one actuating mechanism for actuating the pair of tissue-approximating arms and/or the fastener deforming mechanism. The tissue fastening assembly is configured to provide a way for the tissues to enter into tissue capturing zones. The actuating mechanism is configured to actuate the pair of tissue approximating arms and/or the fastener deforming mechanism in order to deform the at least one fastener and capture the tissue for closing a wound.

### FIELD OF INVENTION

The invention relates to medical devices, particularly a surgical fastener and a device for applying such fasteners to close wounds.

### BACKGROUND

Wound closure has been in existence for many years in the practice of medicine. Although wound closure typically is associated with suturing the wound, many materials have been used over time. Wound closure techniques have evolved significantly and now range from simple sutures to adhesive compounds, and techniques have also improved. Multiple techniques can be used for wound closure. These include sutures, surgical fasteners or staples, and adhesives.

Wound closures are usually done with sutures, staplers, adhesives, or strips. Closure with sutures is generally time-consuming, tedious, and not easy to perform, for certain closure techniques. Another disadvantage of using a suture is possible strangulation and necrosis of tissue due to the high pressure exerted by the relatively small surface area of the suture and over-tightening of the suture in order to tie the suture snugly. Sutures are of two types: non-biodegradable and biodegradable. The disadvantages of using biodegradable sutures include very early loss of tensile strength of the sutures, inflammation by the degradation products of the suture, and subsequent granulomas in the tissue. The non-biodegradable sutures need to be removed after some days; if kept for longer periods, they may lead to scarring of the tissue.

Surgical staplers are increasingly used in both human and veterinary medicine as an alternative to sutures for closing wounds and incisions. They require less skill to use, allow for quicker and easier removal, reduce the risk of errors, and often provide better cosmetic results compared to traditional sutures. Additionally, stapling shortens procedure time, lowering the risk of postoperative infection and offering economic benefits. However, the conventional metal staplers have some drawbacks.

Further, the prior art document U.S. Pat. No. 3,643,851A discloses a surgical instrument for applying sterilized staples from a disposable staple-carrying cartridge to the disunited skin of a patient to effect a joining of the skin. The instrument consists of an anvil adapted to lie flush with the skin, a disposable cartridge housing a plurality of staples that are to be folded around the anvil, and a pusher for bending the staples around the anvil. The pusher is U-shaped with chamfers on the arms thereof to effect the bending with a minimum of force. A gas-powered unit serves to eject and form the staples neatly and uniformly. The novel disposable gas cartridge is also disclosed.

In these settings, the incision tends to make a clean, straight cut, with the opposing sides of the incision having consistent and non-jagged surfaces. Typically, stapling of a skin opening, for example, is accomplished by manually approximating the opposing sides of the skin opening and then positioning the surgical stapler so that a staple will span the opening. The surgical stapler is then manipulated such that the staple is driven into the skin, with one leg being driven into each side of the skin and the cross-member of the staple extending across the opening external to the skin surface. Generally, the legs of the staple are driven into an anvil, causing the staple to deform to retain the skin tissue in a compressed manner within the staple. This process can be repeated along the length of the opening so that the entire incision is held closed during the healing process.

Stapling techniques continue to provide an effective manner of effectuating skin closure, there remains a series of inherent disadvantages in using either of these techniques. The standard technique for both suturing and stapling includes puncturing both the epidermis and dermis. This can result in wound closure having an aesthetically unpleasing appearance on the surface of the skin. The presence of the fastener exposed through the skin surface provides an opportunity for infection and for accidentally catching the fastener and tearing the wound open. In the case of un-absorbable fasteners, further action by a medical professional is necessary to remove the fastener once biological healing is complete. Further, the conventional surgical stapler is more costly and it is not compact and reliable.

To overcome the drawbacks of conventional metal skin stapler, polymeric skin staples are being used for wound closure. Although polymeric staples also have some drawbacks, the biggest drawback of this type of staple is that it has significantly lower tensile strength. Moreover, the currently available staples don't have a closed loop to securely capture the tissue. Most biodegradable polymeric staples are not able to capture the tissue from deep inside the skin layer. This may result in loose connections between the tissue, which may not be able to handle the pressure and increase the chances of the wound opening. Because of this, it is not suitable for high-tension wound closures. The deeper layer must be closed with some other tension-reducing method in order to use the polymeric staplers. Other disadvantages include high cost, highly inflammatory degradation products, and sometimes protrusion of the staple out of the wound.

Many prior art documents disclose the wound closure fastener. The prior art document U.S. Pat. No. 7,112,214B2 discloses a dynamic bio-absorbable staple for use with a wound in living human tissue having opposed sides, the staple comprising a bio-absorbable staple body including a pair of staple arms operably joined at a shoulder portion by a backspin, each arm further including an elbow portion having an inwardly projecting cleat, the staple arms, the inwardly projecting cleats and the back-span defining an internal tissue capture zone; and each shoulder portion including an interior shoulder angle generally defined by the back-span and the staple arm, the shoulder portion constructed so that the interior shoulder angle is between 70°-100° in the first position at an insertion time, the interior shoulder angle transitioning to between 120°-180° in a second deformed position at a second time after the insertion time in response to lateral forces naturally exerted by the opposing sides of the wound and deformation of at least the shoulder portions of the bio-absorbable staple body caused by polymeric creep and adsorption of the bio-absorbable staple body in the living human tissue.

Conventional metal surgical staplers often cause patient discomfort during healing, leave scars, and require removal if non-biodegradable. While polymeric staples address some issues, they face critical limitations: low tensile strength, lack of closed-loop designs to securely grip deeper tissue layers, and an inability to withstand high-tension wounds, risking reopening. Additional challenges include reliance on secondary tension-reduction methods, inflammatory degradation byproducts, high costs, and occasional staple protrusion from wounds. These shortcomings highlight the need for improved biodegradable solutions that balance strength, secure tissue capture, and minimal adverse effects.

Therefore, there is a need for a surgical fastener and a device for applying such fasteners easily and effectively for closing a wound. There is also a need for a surgical fastener that is made of a biodegradable material that offers an enhanced balance of strength, biocompatibility, and biodegradability. There is also a need for a surgical fastener that has a unique design and desired mechanical properties, which is suitable for closing the deep layer in conventional bi-layer wound closures or can act as a single-layer wound closure technique, without the disadvantages of currently available wound closure modalities. Further, there is also a need for a surgical fastener and a device that offer ease of handling and cost-effective wound closure tool.

### SUMMARY OF THE INVENTION

The following presents a simplified summary of one or more embodiments of the present disclosure to provide a basic understanding of such embodiments. This summary is not an extensive overview of all contemplated embodiments and is intended to neither identify key nor critical elements of all embodiments, nor delineate the scope of any or all embodiments.

The present disclosure, in one or more embodiments, relates to a novel surgical fastener and a device that is capable of applying such fasteners easily and effectively for holding the tissue together. The surgical device that applies the fasteners into dermal tissue particularly, thus acts as a deep dermal wound closure device.

An embodiment of the first aspect, a surgical device is disclosed that is configured to apply the fasteners efficiently and effectively for securing tissue. The surgical device comprises a tissue fastening assembly, a pair of tissue-approximating arms, a fastener deforming mechanism, a plurality of fasteners and at least one actuating mechanism for actuating the pair of tissue-approximating arms and/or the fastener deforming mechanism.

In one embodiment, the tissue fastening assembly having a pair of slits opposite to each other at the lower end of the tissue fastening assembly. The pair of slits is adapted to allow the tissue to enter into tissue capturing zones.

In one embodiment, the tissue fastening assembly comprises a pair of prongs. The pair of prongs is extended from the upper end of the tissue fastening assembly. The pair of prongs is situated just behind their respective slits in the tissue fastening assembly. The pair of prongs is configured to guide the deformation of the respective arms of the fastener.

In one embodiment, a pair of tissue-approximating arms is positioned opposite to each other corresponding to the respective slits. Each tissue-approximating arm comprises a ridge at the lower end. The ridge of each tissue approximating arm is adapted to push optimal amount of the tissue into the respective tissue capturing zones of the tissue fastening assembly.

In one embodiment, the fastener deforming mechanism is mostly located at the lower end of the tissue fastening assembly.

In one embodiment, the plurality of fasteners having two tissue capturing zones opposite to each other significantly in a single plane. Each tissue capturing zone is formed by a pair of arms. Each arm of the pairs of arms having two or more segments. At least two arms of the pairs of arms from either or the same tissue-capturing zone are connected at their respective bases, thereby forming a dual-axis symmetry substantially in the same plane. In one embodiment, the fasteners are made of a composition that contains biodegradable metal or metals in the form of an alloy.

In one embodiment herein, the one or more segments of the arms of the pairs of arms in the same tissue-capturing zone are configured to move towards each other for penetrating and capturing the tissue in their respective tissue-capturing zones when the fastener undergoes deformation upon receiving forces by a surgical fastening device. In one embodiment herein, the deformation of the fasteners is evenly distributed across the deformed length of each arm of the pairs of arms instead of at a localized single point.

In one embodiment herein, at least one segment of each arm of the pairs of arms comprises a penetrative tip, which is configured to penetrate into the tissue. The penetrative tips of the arms of the pairs of arms in the same tissue-capturing zones are configured to face inwardly towards each other, forming a gap through which the tissue enters into the respective tissue-capturing zones.

In one embodiment herein, at least two arms of the pairs of arms from either or the same tissue-capturing zone are connected at their respective bases and are connected to the rest of the fastener by a connecting member such that it maintains the dual-axis symmetry substantially in the same plane.

In one embodiment, at least one actuating mechanism is configured to actuate the pair of tissue-approximating arms and/or the fastener deforming mechanism, thereby facilitating the penetration and holding of the tissue with the fasteners.

In one embodiment at least one actuating mechanism comprises a pair of legs.

In one embodiment at least one actuating mechanism comprises a pair of elongated legs.

The pair of legs is extended from the upper part of the surgical device corresponding to their respective arms of the pair of tissue-approximating arms. The pair of legs are configured to activate the pair of tissue approximating arms, thereby enabling the pair of tissue approximating arms to capture the tissue. The pair of elongated legs is functionally mated with a plurality of pushers, downwardly extended from the upper part of the surgical device. The pair of elongated legs is configured to activate the plurality of pushers to deform the pairs of arms of the fasteners in a desired manner.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

The accompanying drawings, which are incorporated in and constitute a part of the specification, illustrate an embodiment of the invention, and, together with the description, explain the principles of the invention.

FIG. 1A illustrates an exploded view of the surgical device, in accordance with embodiments of the invention.

FIG. 1B illustrates a front view of the surgical device, in accordance with embodiments of the invention.

FIG. 2 illustrates a bottom view of the tissue fastening assembly, in accordance with embodiments of the invention.

FIG. 3A illustrates a cross sectional view of at least one undeformed fastener resting on the floor of the tissue fastening assembly, in accordance with embodiments of the invention.

FIG. 3B illustrates a perspective view of an undeformed fastener, in accordance with embodiments of the invention.

FIG. 3C illustrates a perspective view of a fastener in a deformed state after applying to the tissue, in accordance with embodiments of the invention.

FIG. 4 illustrates a detailed view of the actuating mechanism, in accordance with embodiments of the invention.

FIGS. 5A-5B illustrate detailed views of the surgical device, in accordance with embodiments of the invention.

FIG. 6 illustrates a schematic view of at least one tissue-approximating arm, in accordance with embodiments of the invention.

FIG. 7 illustrates a perspective view of the plurality of pushers of the surgical device, in accordance with embodiments of the invention.

FIG. 8 illustrates a cross sectional view of the plurality of pushers in connection with the floor of the tissue fastening assembly, in accordance with embodiments of the invention.

### DETAILED DESCRIPTION

Reference will now be made in detail to the present preferred embodiments of the invention, examples of which are illustrated in the accompanying drawings. Wherever possible, the same reference numerals are used in the drawings and the description to refer to the same or like parts.

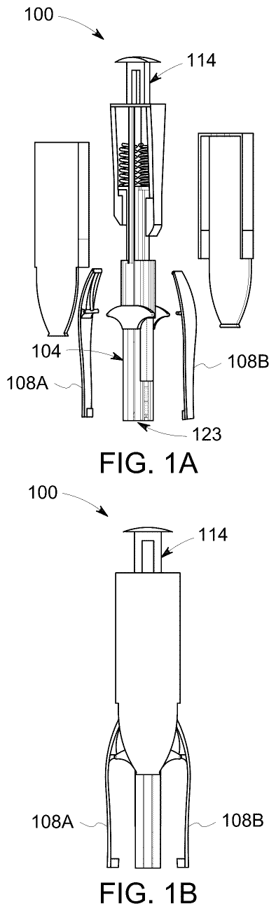

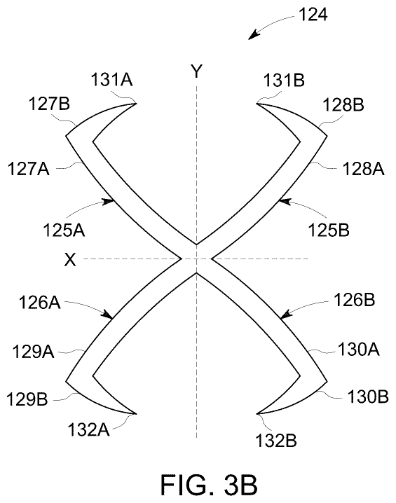

FIG. 1A refers to an exploded view of the surgical device 100, according to one embodiment of the invention. FIG. 1B refers to a front view of the surgical device 100. The surgical device 100 applies the fasteners to dermal tissue particularly and acts as an alternative to sutures for closing the deep layer of a surgical wound. The surgical device 100 is quick and convenient when compared to sutures, thereby saving precious time and effort required in the operation room. The surgical device 100 with the fasteners 124 (as shown in FIG. 3A) offers ease of handling and cost-effective wound closure tool.

In one embodiment herein, the surgical device 100 comprises a tissue fastening assembly 104, a pair of tissue-approximating arms (108A, 108B), a plurality of fasteners 124, at least one actuating mechanism 114 for actuating the pair of tissue-approximating arms (108A, 108B) and/or the fastener deforming mechanism 123.

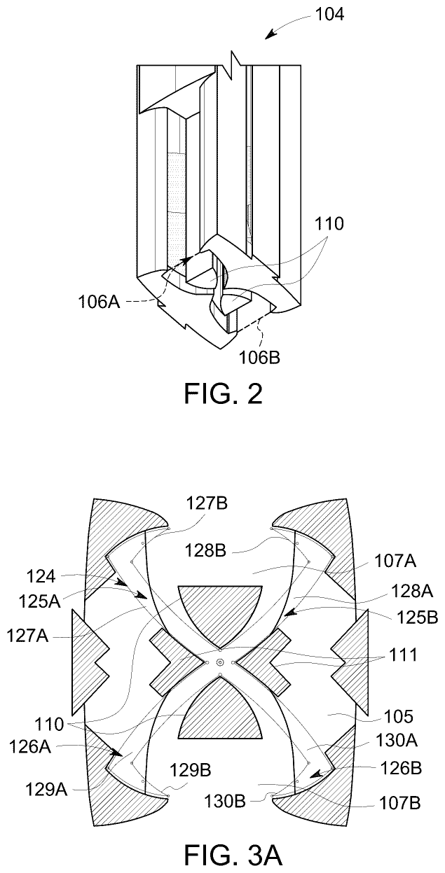

The tissue fastening assembly 104 having a pair of slits (106A, 106B) (as shown in FIG. 2) opposite to each other at the lower end of the tissue fastening assembly 104. The pair of slits (106A, 106B) is adapted to allow the tissue to enter into respective tissue capturing zones (107A, 107B) (as shown in FIG. 3A) of the tissue fastening assembly 104 and the fastener 124.

The pair of tissue-approximating arms (108A, 108B) is positioned opposite to each other corresponding to the respective slits (106A, 106B) of the tissue fastening assembly 104. The pair of tissue-approximating arms (108A, 108B) is configured to move in order to hold the tissue. In one embodiment herein, each tissue-approximating arm (108A, 108B) comprises a ridge 109 at the lower end. The ridge 109 of the pair of tissue-approximating arms (108A, 108B) is adapted to push optimal amount of the tissue into respective tissue capturing zones of the tissue fastening assembly and the fastener 124. The fastener deforming mechanism 123 is mostly located at the lower end of the tissue fastening assembly 104. The fastener deforming mechanism 123 is configured to deform the plurality of fasteners. In some embodiments, the surgical device 100 is used to close a surgical wound with the fasteners 124.

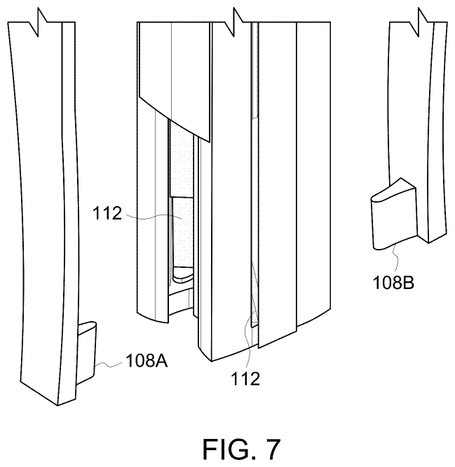

In one embodiment herein, at least one actuating mechanism 114 is operably positioned in connection with the pair of tissue-approximating arms (108A, 108B) and/or the fastener deforming mechanism 123 (as shown in FIG. 7). The actuating mechanism 114 is adapted to actuate the pair of tissue approximating arms (108A, 108B) and/or the fastener deforming mechanism 123 thereby facilitating the penetration and holding of the tissue with the fasteners 124 (as shown in FIG. 3A).

According to one embodiment of the invention, FIG. 2 refers to a bottom view of the tissue fastening assembly 104 of the surgical device 100. In one embodiment herein, the tissue fastening assembly 104 comprises an indicator, which aids in capturing the ideal amount of the tissue. The tissue fastening assembly 104 comprises a pair of prongs 110. The pair of prongs 110 extends from the upper end of the tissue fastening assembly 104. The pair of prongs 110 is situated just behind their respective slits (106A, 106B) in the tissue fastening assembly 104. The pair of prongs 110 is configured to guide the deformation of the respective arms (125A, 125B, 126A, and 126B) of the plurality of the fastener 124. The other pair of prongs 111 is positioned within the tissue fastening assembly 104. The other pair of prongs 111 is configured to hold and stabilize the fasteners 124 while deforming.

According to one embodiment of the invention, FIG. 3A refers to a cross sectional view of at least one undeformed fastener 124 resting on floor 105 of the tissue fastening assembly of the surgical device 100. The fasteners 124 are placed on one another and stacked vertically on the floor 105 of the tissue fastening assembly 104. The portions of the fasteners 124 are mirror images of each other through both the horizontal plane and the vertical plane which are formed by a horizontal axis X and a vertical axis Y passing through the center of the fastener 124. The plurality of fasteners 124 having two tissue capturing zones opposite to each other substantially in a single plane. The fasteners 124 are configured to be deformed and capture the tissue. In one embodiment herein, the plurality of fasteners 124 are made of a composition that contains biodegradable metal or metals in the form of an alloy. The at least one of the biodegradable metals could have significant growth-prompting and anti-bacterial properties.

According to one embodiment of the invention, FIG. 3B refers to a perspective view of the undeformed fastener 124. In one embodiment herein, each tissue capturing zone is formed by a pair of arms (125A, 125B, 126A, and 126B). Each arm (125A, 125B, 126A, and 126B) of the pairs of arms (125A, 125B, 126A, and 126B) having two or more segments (127A, 127B, 128A, 128B, 129A, 129B, 130A, and 130B). At least two arms (125A, 125B, 126A, and 126B) of the pairs of arms (125A, 125B, 126A, and 126B) from either or the same tissue-capturing zone (134, 136) (as shown in FIG. 3C) are connected at their respective bases, thereby forming a dual-axis symmetry substantially in the same plane. Each arm of the pairs of arms (125A, 125B, 126A, and 126B) in the same tissue-capturing zone (134, 136) having two or more segments (127A, 127B, 128A, 128B, 129A, 129B, 130A, and 130B), which is configured to move towards each other for penetrating and capturing the tissue (12, 14) (as shown in FIG. 3C).

In one embodiment herein, at least one segment (127B, 128B, 129B, and 132B) of each arm of the pairs of arms (125A, 125B, 126A, and 126B) comprises a penetrative tip (131A, 131B, 132A, and 132B), which is configured to penetrate into the tissue (12, 14). The penetrative tips (131A, 131B, 132A, and 132B) of the arms of the pairs of arms (125A, 125B, 126A, and 126B) in the same tissue-capturing zones (134, 136) are configured to face inwardly towards each other, forming a gap through which the tissue (12, 14) enters into the respective tissue-capturing zones (134, 136).

According to one embodiment of the invention, FIG. 3C refers to a top view of the fastener 124 in a deformed state after applying to the tissue. In one embodiment herein, the two or more segments (127A, 127B, 128A, 128B, 129A, 129B, 130A, and 130B) of the arms of the pairs of arms (125A, 125B, 126A, and 126B) in the same tissue-capturing zone (134, 136) are configured to move towards each other for penetrating and capturing the tissue (12, 14) in their tissue-capturing zones (134, 136) when the fastener 124 undergoes deformation upon receiving forces by the surgical fastening device. In one embodiment herein, the plastic deformation of the fastener 124 is evenly distributed across the deformed length of each arm of the pairs of arms (125A, 125B, 126A, and 126B) instead of a localized single point.

In one embodiment herein, at least two arms of the pairs of arms (125A, 125B, 126A, and 126B) from either or the same tissue-capturing zone (134, 136) are connected at their respective bases and are connected to the rest of the fastener 124 by a connecting member such that it maintains the dual-axis symmetry substantially in the same plane.

In one embodiment herein, the fastener 124 is capable of capturing optimal amounts of the tissue (12, 14) in their respective tissue-capturing zones (134, 136), which provides high tensile strength for holding the wound. In one embodiment herein, the one or more segments (127A, 127B, 128A, 128B, 129A, 129B, 130A, and 130B) of the arms of the pairs of arms (125A, 125B, 126A, and 126B) in the same tissue-capturing zone (134, 136) make a closed loop by which the tissue (12, 14) are held in place.

The fasteners 124 are engineered for safe, effective wound closure with enhanced tensile strength, uniform stress distribution, and firm tissue grip, reducing the risk of slippage, necrosis, and tissue damage. Their design promotes wound eversion, simplifies application without metal components, and supports healing through growth-promoting, anti-bacterial, and anti-inflammatory properties. Customizable degradation rates and even deformation across the arms further enhance reliability, biocompatibility, and clinical versatility.

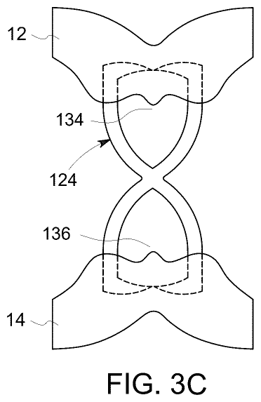

According to one embodiment of the invention, FIG. 4 refers to a detailed view of the actuating mechanism 114 for actuating the pair of tissue-approximating arms (108A, 108B) (as shown in FIG. 1) and/or the fastener deforming mechanism 123 (as shown in FIG. 1A). In one embodiment herein, the actuating mechanism 114 combined comprises a head 116, a pair of legs (120A, 120B), a pair of elongated legs (121A, 121B). The pair of legs (120A, 120B) extends from the upper part of the surgical device 100 corresponding to their respective arms of the pair of tissue-approximating arms (108A, 108B). The pair of legs (120A, 120B) is configured to actuate the pair of tissue-approximating arms (108A, 108B), thereby enabling the pair of tissue-approximating arms (108A, 108B) to capture the tissue. At least one leg 120A from the pair of legs (120A, 120B) is designed shorter than another leg 120B for sequential actuation of the pair of tissue-approximating arms (108A, 108B). The pair of elongated legs (121A, 121B) is functionally mated with a plurality of pushers 112 (as shown in FIG. 7), and downwardly extended from the upper part of the surgical device 100. The pair of elongated legs (121A, 121B) is configured to activate the plurality of pushers 112 to deform the pairs of arms (125A, 125B, 126A, and 126B) (as shown in FIG. 3A) of the fasteners 124 in a desired manner. The plurality of pushers 112 is configured to deform the arms of the pairs of arms (125A, 125B, 126A, and 126B) of the plurality of fasteners 124.

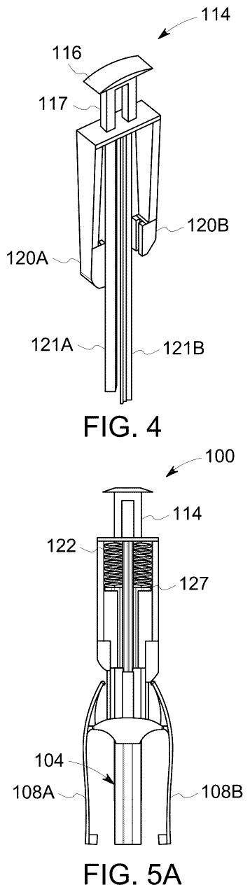

The head 116 is connected with a neck portion 117, which downwardly extends from the head 116. The head 116 is adapted to allow the user to manually press against an elastic member 122 (as shown in FIG. 5A). The elastic member 122 is supported by a support member 127 (as shown in FIG. 5A). The elastic member 122 is configured to provide a constant downward force on the fasteners 124 and an upward force on the actuating mechanism 114. The profile of the elongated legs (121A, 121B), when seen from the cross section, is designed in a way to exert forces on the pushers 112 in a desired manner.

According to another exemplary embodiment of the invention, FIGS. 5A-5B refer to detailed views of the surgical device 100. In one embodiment herein, the surgical device 100 before actuating the pair of tissue-approximating arms (108A, 108B), is shown in the FIG. 5A. In another embodiment herein, the surgical device 100 after actuating the pair of tissue-approximating arms (108A, 108B), is shown in the FIG. 5B.

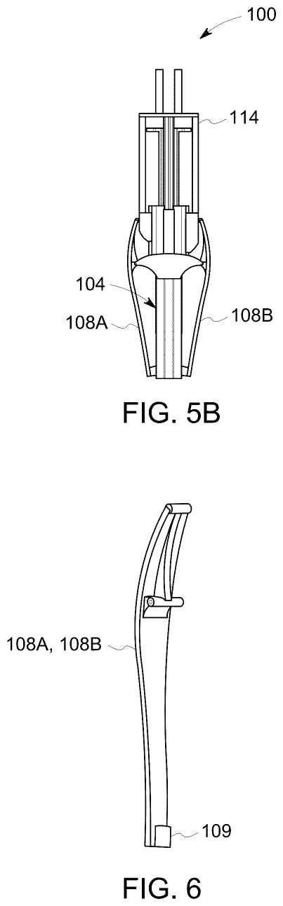

According to another exemplary embodiment of the invention, FIG. 6 refers to a schematic view of at least one tissue-approximating arm (108A, 108B). In one embodiment herein, the lower end of the lower part of the tissue approximating arms (108A, 108B) comprise a ridge 109 along the longitudinal axis, which extends from the tissue-approximating arms (108A, 108B) towards the respective slits (106A, 106B) (as shown in FIG. 2) of the of the tissue fastening assembly 104. The length and the curvature of the ridge 109 is designed to push optimal amount of the tissue into the tissue capturing zones (107A, 107B) (as shown in FIG. 3A) of the tissue fastening assembly 104 in a desired manner.

According to another exemplary embodiment of the invention, FIG. 7 refers to a perspective view of the plurality of pushers 112 and the pair of tissue-approximating arms (108A, 108B) of the surgical device 100. In one embodiment herein, the each tissue-approximating arm (108A, 108B) makes a pivot joint with the tissue fastening assembly 104, which acts as a fulcrum for the upper and the lower part of the tissue-approximating arms (108A, 108B). Both the tissue-approximating arms (108A, 108B) have an identical, specific curvature when viewed from the front, and they are made of a material with specific elasticity designed to capture optimal amount of tissue without causing damage to the tissue. The upper end of each tissue-approximating arm (108A, 108B) comes into contact with the pair of legs (120A, 120B) when activated, causing the lower end of each tissue-approximating arm (108A, 108B) to move inward towards their respective slits (106A, 106B) of the tissue fastening assembly 104.

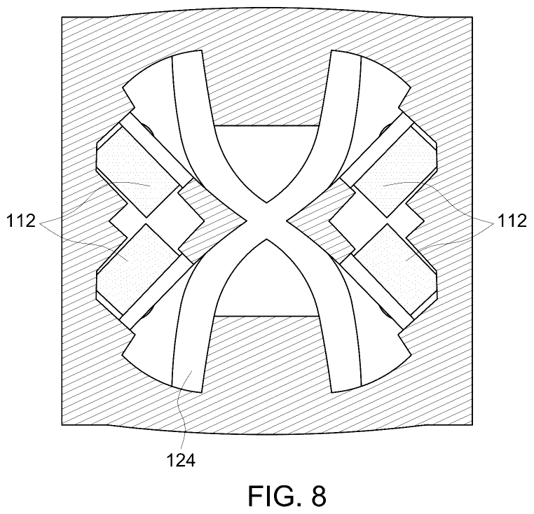

According to another exemplary embodiment of the invention, FIG. 8 refers to a cross sectional view of the plurality of pushers 112 in connection with the floor 105 (as shown in FIG. 3A) of the tissue fastening assembly 104. In one embodiment herein, the plurality of pushers 112 is operably positioned at the bottom end of the tissue fastening assembly 104. The plurality of pushers 112 is configured to deform the pairs of arms (125A, 125B, 126A, and 126B) of the plurality of fasteners 124 upon activation. The plurality of pushers 112 is in a vertical orientation, making an angle of less than 20 degree to the vertical axis of the surgical device 100 and are mostly parallel to at least one corresponding arm (125A, 125B, 126A, and 126B) of the fastener 124 in the horizontal plane of the tissue fastening assembly 104. The tip of each pusher in the plurality of pushers 112 is bent inwards usually normal to the pusher 112 itself. The tip of each pusher in the plurality of pushers 112 sits in close proximity to at least one corresponding arm (125A, 125B, 126A, and 126B) of the pairs of arms of the plurality of the fasteners 124. The tip of the plurality of pushers 112 has a curve, which is designed to deform at least one arm (125A, 125B, 126A, and 126B) of the fastener 124 in a desired way when the fastener deformation mechanism is activated.

The surgical device 100 efficiently applies biodegradable fasteners 124 for deep dermal wound closure, offering precise tissue alignment and consistent engagement with opposing tissue-capturing zones on the same plane. It simplifies bi-layer closure, reduces operating time, and serves as a faster, more convenient alternative to sutures. Its compact, user-friendly, and cost-effective design enhances surgical efficiency and supports natural healing.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principles of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

It will readily be apparent that numerous modifications and alterations can be made to the processes described in the foregoing examples without departing from the principles underlying the invention, and all such modifications and alterations are intended to be embraced by this application.

The claimed invention is:

1. A surgical device, comprising:
a tissue fastening assembly having a pair of slits, which are opposite to each other at the lower end of the tissue fastening assembly, wherein the pair of slits is adapted to allow tissue to enter into respective tissue capturing zones of the tissue fastening assembly;
a pair of tissue-approximating arms positioned opposite to each other corresponding to the respective slits from the pair of slits;
a fastener deforming mechanism operably positioned at the lower end of the tissue fastening assembly;
a plurality of fasteners, wherein each of the fastener having two tissue-capturing zones opposite to each other substantially in a single plane, wherein each of the tissue-capturing zone is defined by a pair of arms, where each of the arm of the pair of arms having at least two segments,
wherein at least two arms of the pairs of arms from either or the same tissue-capturing zone are connected at their respective bases, thereby forming a dual-axis symmetry substantially in the single plane in an ideal unfastened condition; and
at least one actuating mechanism configured to actuate the pair of tissue-approximating arms and/or the fastener deforming mechanism, thereby facilitating penetration and secure holding of the tissue by the fasteners as they undergo deformation during deployment.

2. The surgical device of claim 1, wherein the tissue fastening assembly comprises a plurality of pushers that is functionally positioned at the lower end of the tissue fastening assembly, wherein the plurality of pushers are configured to deform the pairs of arms of each of the plurality of fasteners.

3. The surgical device of claim 1, wherein the at least one actuating mechanism comprises a pair of legs extending from an upper part of the at least one actuating mechanism, wherein each of the pair of legs is operatively associated with the respective tissue-approximating arm of the pair of tissue-approximating arms.

4. The surgical device of claim 3, wherein the pair of legs is configured to respectively actuate the pair of tissue-approximating arms, thereby enabling the pair of tissue-approximating arms to capture the tissue.

5. The surgical device of claim 1, wherein the at least one actuating mechanism comprises a pair of elongated legs downwardly extended from the upper part of the at least one actuating mechanism, wherein the pair of elongated legs is operatively engaged with the plurality of pushers.

6. The surgical device of claim 5, wherein the pair of elongated legs is configured to activate the plurality of pushers to deform the pairs of arms of each of the fastener in a desired manner.

7. The surgical device of claim 1, wherein the tissue fastening assembly comprises a pair of prongs
which is extended from an upper end of the tissue fastening assembly, wherein the pair of prongs is situated behind the pair of slits, respectively, in the tissue fastening assembly, and is configured to guide the deformation of the pairs of arms of each of the plurality of fasteners.

8. The surgical device of claim 1, wherein each of the tissue-approximating arm comprises a ridge at the lower end, wherein the ridge of each of the tissue-approximating arm is adapted to push optimal amount of the tissue into the respective tissue capturing zones of the tissue fastening assembly.

9. The surgical device of claim 1, wherein the one or more segments of each arm of the pairs of arms in the same tissue-capturing zone are configured to move towards each other for penetrating and capturing the tissue in their respective tissue-capturing zones when the at least one fastener undergoes deformation upon actuation of the at least one actuating mechanism.

10. The surgical device of claim 1, wherein the tissue fastening assembly or the tissue approximating arms comprises an indicator, which aids in capturing the ideal amount of the tissue.

11. A surgical tissue fastener, comprising:
two tissue-capturing zones opposite to each other substantially in a single plane, wherein the each tissue-capturing zone is formed by a pair of arms, wherein each arm of the pairs of arms having two or more segments,
wherein at least two arms of the pairs of arms from either or the same tissue-capturing zone are connected at their respective bases, thereby forming a dual-axis symmetry substantially in the same plane in an ideal unfastened condition,
wherein the one or more segments of the arms of the pairs of arms in the same tissue-capturing zone are configured to move towards each other for penetrating and capturing tissue in their respective tissue-capturing zones when the fastener undergoes deformation upon receiving forces by a surgical fastening device.

12. The fastener of claim 11, wherein at least one segment of each arm of the pairs of arms comprises a penetrative tip, which is configured to penetrate into the tissue.

13. The fastener of claim 12, wherein the penetrative tips of the arms of the pairs of arms in the same tissue-capturing zones are configured to face inwardly towards each other, forming a gap through which the tissue enters into the respective tissue-capturing zones.

14. The fastener of claim 11, wherein the deformation of the fastener is evenly distributed across the deformed length of each arm of the pairs of arms instead of a localized single point.

15. The fastener of claim 11, wherein at least two arms of the pairs of arms from either or the same tissue-capturing zone are connected at their respective bases and are connected to the rest of the fastener by a connecting member such that it maintains the dual-axis symmetry.

16. The fastener of claim 11, wherein the fastener is made of a composition that contains biodegradable metal or metals in the form of an alloy.
